(19) United States
(12) Patent Application Publication
(10) Pub. No.: US 2018/0169242 A1
(43) Pub. Date: Jun. 21, 2018

(54) PHARMACEUTICAL COMBINATION FOR CANCER TREATMENT AND THE THERAPEUTIC USE THEREOF

(71) Applicant: Shiner Pharm Corp., New Taipei (TW)

(72) Inventor: SZU-YUAN WU, Pingtung (TW)

(21) Appl. No.: 15/838,280

(22) Filed: Dec. 11, 2017

Related U.S. Application Data
(60) Provisional application No. 62/436,364, filed on Dec. 19, 2016.

(51) Int. Cl. A61K 41/00; A61K 31/155; A61K 31/405; A61N 5/10

(52) U.S. Cl. CPC A61K 41/0038; A61K 31/155; A61N 2005/1098; A61N 5/10; A61K 31/405

(57) ABSTRACT

A pharmaceutical combination for sensitizing a subject to a cancer treatment comprises an amount of metformin and an amount of statin. The amount of metformin and the amount of statin together constitute an effective amount of the pharmaceutical combination. The statin is selected from a group consisting of atorvastatin, fluvastatin, lovastatin, pravastatin, rosuvastatin, and simvastatin. The cancer treatment is preferably a radiotherapy. The pharmaceutical combination may be included in a pharmaceutical composition. A method of treating cancer is also provided. The method comprises administering the pharmaceutical combination to a subject and delivering an effective dose of radiation to the subject.

### CROSS-REFERENCE TO RELATED APPLICATION

The present application claims priority of U.S. provisional patent application No. 62/436364, filed on Dec. 19, 2016, the entirety of which is incorporated herein by reference.

### FIELD

The present invention herein generally relates to a pharmaceutical combination and its use in cancer therapy. More particularly, the present invention relates to a pharmaceutical combination that comprises metformin and statin and a therapeutic use thereof.

### BACKGROUND

Chemotherapy and radiotherapy are two of the most common cancer treatments. These therapies kill cancer cells by damaging the DNA or interfering the cell cycle. However, there are many reports show that chemotherapy and radiotherapy may cause harmful side effects to the patients, because they may cause non-specific damages to normal tissues. Therefore, there is a need to find a way to sensitize cancer cells to cancer treatments and reduce the therapeutic doses of harmful cancer therapies.

Metformin is mainly known for its anti-hyperglycemic activity and is widely used in treatments of non-insulin-dependent diabetes. In the case of insulin-dependent diabetes, metformin is also administered to the patients in combination with insulin.

Statins are hydroxymethylglutaryl coenzyme A (HMG-CoA) reductase inhibitors that inhibit HMG-CoA reductase, and therefore inhibit or interfere with synthesis of cholesterol. Inhibition of cholesterol synthesis can lead to reduction in blood cholesterol levels. Conventionally, these drugs are used in patients to lower low density lipoprotein (LDL) cholesterol.

### SUMMARY OF THE INVENTION

The present disclosure provides that the combination of metformin with statin leads to a significant enhancement in efficacy of cancer treatments. More particularly, the present disclosure provides a pharmaceutical combination that includes metformin and statin and is used as a sensitizer for cancer treatments. Using the combination of metformin with statin in cancer therapy has never been described in the prior art.

A cancer therapeutic combination according to an embodiment of the present disclosure includes metformin and a statin. Preferably, the cancer therapeutic combination comprises an amount of metformin, an amount of statin, and a pharmaceutically acceptable carrier, diluent, excipient or a combination thereof.

A cancer therapeutic combination according to an embodiment of the present disclosure includes metformin and a first pharmaceutically acceptable carrier, diluent, excipient or a combination thereof; and statin and a second pharmaceutically acceptable carrier, diluent, excipient or a combination thereof.

A combination of metformin and statin according to an embodiment of the present disclosure is to be used as a cancer therapy sensitizer.

### BRIEF DESCRIPTION OF THE DRAWINGS

Implementations of the present invention will now be described, by way of example only, with reference to the attached figures.

FIG. 1A is a bar chart of experimental results indicating that administration of metformin alone, statin alone, or the combination of metformin and statin did not show local tumor-suppressive effect in Lewis lung carcinoma animal model.

FIG. 1B is a bar chart of experimental results indicating that administration of the combination of metformin and statin enhances the therapeutic effect of radiotherapy and shows local tumor-suppressive effect, while neither combining radiotherapy with metformin alone or with statin alone could suppress local tumor growth.

FIG. 2 is a bar chart of experimental results indicating that administration of the combination of metformin and statin suppresses radiotherapy enhanced metastasis in Lewis lung carcinoma animal model.

### DETAILED DESCRIPTION OF THE PREFERRED EMBODIMENTS

It will be understood by those of ordinary skill in the art that the examples described herein can be practiced without these specific details. In other instances, methods, procedures and components have not been described in detail so as not to obscure the related relevant feature being described. The description is not to be considered as limiting the scope of the examples described herein.

Several definitions that apply throughout this disclosure will now be presented.

As used herein the specification, “a” or “an” may mean one or more. As used herein in the claim(s), when used in conjunction with the word “comprising”, the words “a” or “an” may mean one or more than one. As used herein “another” may mean at least a second or more.

The term “comprising” when used herein, means “including, but not necessarily limited to”; it specifically indicates open-ended inclusion or membership in the so-described combination, group, series and the like.

The terms “pharmaceutical combination” or “combination” are used interchangeably herein and refers to co-administration of multiple therapeutic agents in a substantially simultaneous manner, such as in a single capsule or dosage device having a fixed ratio of these active agents or in multiple, separate capsules or dosage devices that can be taken together contemporaneously. In the present disclosure, the “pharmaceutical combination” or “combination” refer to a combination of metformin and statin.

By the term “treat” as in “treat a subject,” is meant to give medical aid to such subject especially, for the purposes of preventing the development of, or preventing the worsening of an undesired physiological or medical condition, or for the purposes of ameliorating such condition in such subject, either human or animal. Unless otherwise stated, the term “treat” is not limited to any particular length of time or to any particular level of dose. The term “treat” do not necessarily imply 100% or complete treatment.

The terms “sensitizer for cancer treatment” or “cancer therapy sensitizer” are used interchangeably herein, and refer to the composition containing an effective amount of two materials and a pharmaceutically acceptable carrier, diluent, excipient or a combination thereof. For example, the aforementioned composition can be applied before, during or both before and during the cancer therapy to improve or enhance the effect of one or more effective amount of therapeutically active composition upon a cancer or a tumor in an individual in need, and then achieve the effect of eliminating, inhibiting, improving, alleviating or preventing a cancer and its symptoms; retarding, prohibiting, reversing the rate of tumor proliferation; or the medical effects similar to the aforementioned effects. Wherein the composition mentioned above can be a combination of metformin and statin. A cancer therapy method can be, for example, radiotherapy to irradiate the lesion of a cancer or a tumor with an effective dose of radiation, or chemotherapy to provide an effective amount of therapeutically active composition.

The terms “composition” or “pharmaceutical composition” are used interchangeably herein and refers to compositions that usually comprise an excipient, such as a pharmaceutically acceptable carrier that is conventional in the art and that is suitable for administration to a subject. Such compositions can be specifically formulated for administration via one or more of a number of routes, including but not limited to, oral, ocular and nasal administration and the like.

The term “formulation” used herein refers to an applicable unit of a pharmaceutical product, such as a capsule, a tablet, or an emulsion, prepared according to a specific pharmaceutical formulating procedure. The pharmaceutical formulating procedure refers to a process of combining all ingredients that determines the characteristics, such as safety, medical effects, or production cost, of the pharmaceutical product.

The terms “effective amount” used herein means the dose or effective amount to be administered to a subject. The dose or effective amount refers to an amount of the materials or the compounds mentioned, which can effectively improve the effects of a cancer therapy, such as cancer cell killing, reduction of tumor size, inhibition or retardation of cancer progression, reduction of cancer relapse rate, preventing the formation of a cancer or a tumor. The dose or effective amount to be administered to a subject and the frequency of administration to the subject can be readily determined by one of ordinary skill in the art by the use of known techniques and by observing results obtained under analogous circumstances. In determining the effective amount or dose, a number of factors are considered by the diagnostician attending to the case, including but not limited to, the potency and duration of action of the compounds used, the nature and severity of the illness to be treated, as well as the sex, age weight, general health and individual responsiveness of the subject to be treated, and other relevant circumstances. And “an effective dose” refers to the dose value of the radiation mentioned, which can also accomplish the effects aforementioned after being absorbed by a living tissue.

The term “pharmaceutically acceptable carrier” used herein means any pharmaceutically acceptable means to mix and/or deliver the targeted delivery composition to a subject. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and is compatible with administration to a subject, for example a human.

The terms “radiotherapy” or “radiation therapy” are used interchangeably herein and refers to the use of ionizing radiation to kill cancer cells and shrink tumors.

The term “chemotherapeutic agent” used herein refers to a chemical or drug used in the treatment of a cancer or a tumor. Such agents are often cytotoxic agents.

The term “subject”, individual”, and “patient” are used interchangeably herein, and refer to an animal such as a mammal. The term “mammal” is intended to encompass a singular “mammal” and plural “mammals,” and includes, but is not limited to: humans, primates such as apes, monkeys, orangutans, and chimpanzees; canids such as dogs and wolves; fields such as cats, lions, and tigers; equids such as horses, cows, donkeys, and zebras, food animals such as cows, pigs, and sheep; ungulates such as deer and giraffes; rodents such as mice, rabbits, rats, hamsters and guinea pigs.

As used herein “fractional doses”, “fractions” or “fractionations” of radiation therapy refer to dividing the total dose of radiation therapy into several smaller doses delivered over a period of time. The total dosage may be fractionated to allow normal cells time to recover, to allow tumor cells that were in a relatively radio-resistant phase of the cell cycle during one treatment to cycle into a sensitive phase of the cycle before the next fraction is given, to allow hypoxic tumor cells to reoxygenate between fractions, or to improve killing of tumor cells. The summed value of individual fractionized dose should add up to about the total dose of radiation therapy prescribed.

The terms “cumulative dose” used herein means the total amount of a radiation given to patient over a period of time.

### Metformin and Statin

The present disclosure is described in relation to a pharmaceutical combination comprising metformin and statin.

The metformin as disclosed herein refers to metformin or a pharmaceutically acceptable salts thereof such as hydrochloride, fumarate, embonate, succinate, chlorophenoxyacetate, hydrobromide, acetate, benzoate, citrate, glycolate, palmoate, aspartate, methanesulphonate, maleate, parachlorophenoxyisobutyrate, formate, lactate, sulphate, tartrate, cyclohexanecarboxylate, hexanoate, octanoate, decanoate, hexadecanoate, octodecanoate, benzenesulphonate, trimethoxybenzoate, paratoluenesulphonate, adamantanecarboxylate, glycoxylate, glutamate, pyrrolidonecarboxylate, naphthalenesulphonate, glucosephosphonate, nitrate, sulphite, dithionate, or phosphate. More particularly, the metformin is in the form of a pharmaceutically acceptable salt selected from the group consisting of hydrochloride, fumarate, embonate, succinate and chlorophenoxyacetate.

In the present disclosure, the statin is selected from the group consisting of atorvastatin, cerivastatin, fluvastatin, itavastatin, lovastatin, mevastatin, pravastatin, pitavastatin, simvastatin, rosuvastatin and chemical derivatives thereof, including the pharmaceutical acceptable salts, solvates, esters and adducts thereof. More particularly, the statin is selected from the group consisting of atorvastatin, fluvastatin, lovastatin, pravastatin, rosuvastatin, and simvastatin.

### The Pharmaceutical Combination

The pharmaceutical combination of metformin and statin disclosed herein is used in cancer treatments. More particularly, the pharmaceutical combination of metformin and statin is used as a sensitizer for cancer treatments, such as chemotherapy or radiotherapy. The preparation of a cancer therapeutic sensitizer is not limited to uniformly mixing metformin and statin; that is, in cancer therapy sensitizers with the same dosage form, metformin and statin can be mixed in other ratios or need not be mixed. For example, a cancer therapy sensitizer can be a tablet or a capsule, wherein part of the tablet or capsule being metformin and another part thereof being statin.

The amount of metformin and the amount of statin together provide a dosage or amount that is sufficient to constitute an effective amount of the combination.

For the combination of metformin and statin to exhibit sensitizing effects, the weight percentage ratio of an amount of metformin or pharmaceutically acceptable salt thereof over an amount of statin or pharmaceutically acceptable salt thereof may vary from 1:1 to 20000:1. It is preferred that the weight ratio of the amount of metformin over the amount of statin administered to the subject is within a range of about 1:1 to about 2000:1, preferably about 1:1 to about 1000:1, or more preferably about 2:1 to about 550:1. For example, the ratio of the amount of metformin over the amount of statin may be, but is not limited to, 2.5:1, 6.25:1, 12.5:1, 16.67:1, 21.25:1, 25:1, 33.33:1, 37.5:1, 42.5:1, 50:1, 62.5:1, 63.75:1, 75:1, 85:1, 100:1, 125:1, 127.5:1, 150:1, 170:1, 200:1, 250:1, 255:1, 300:1, 400:1, 500:1, or 550:1. The ratio of metformin over statin in the pharmaceutical composition according to various embodiments of the present invention will vary if the pharmaceutical composition is to be taken more than once a day, or if it is to be taken only once a day.

The administration dose of metformin and statin may vary when used to treat different species, for example mouse or human. An once a day amount of metformin is generally from 10 mg/kg to 1000 mg/kg for mice. The amount of metformin should be adjusted to 0.8 mg/kg-80 mg/kg when treating a human subject. The most common compositions for human contain 500 mg or 850 mg of metformin and are to be taken twice or three times a day. Thus, if the pharmaceutical compositions of the embodiments of the present invention contain 500 mg or 850 mg of metformin and are to be taken more than once a day, the amount of statin should be adjusted accordingly. An once a day amount of statin is generally from 5 mg/kg to 100 mg/kg for mice. The amount of statin should be adjusted 0.04 mg/kg to 8 mg/kg when treating a human subject. The amount of statin used in the pharmaceutical compositions of the embodiments of the present invention may vary by the specific statin compound used in the pharmaceutical compositions.

The pharmaceutical combination of metformin and statin according to the embodiments of the present invention may be formed in, for example, a solid or liquid oral administration dosage form, such as a tablet, a capsule elixir, or preferably an orally administrable tablet containing an effective amount of the pharmaceutical combination. Of course, the pharmaceutical combination can also be formed as other available dosage forms, such as pills, sachets, granules, powders, chewing gums, suspensions, emulsions, suppositories and solutions.

When used as a cancer therapy sensitizer, there may be a dose unit for the combination of metformin and statin in every formulation; that is, one formulation contains enough dose of the sensitizer for performing sensitizing effect in an individual in need, for facilitating direct administration. In certain examples, every tablet or capsule contains a dose unit of the cancer therapy sensitizer. Needless to say, in other examples of the present invention, one dose unit of the cancer therapy sensitizer can be separated into several sub-dose units or sub-packages, for example, separated into two to three tablets or capsules and packed in the same blister pack.

Pharmaceutical composition of the combination formulated in a manner suitable for administration to humans are known to the skilled in the art. The pharmaceutical composition of the embodiments of the present disclosure may further comprise stabilizers, buffers, etc.

In certain examples, the combination of metformin and statin can further include a pharmaceutically acceptable carrier, diluent, excipient or a combination thereof for preparing a compatible dosage form or prescription form. The pharmaceutically acceptable carrier, diluent or excipient can be, for example, magnesium carbonate, magnesium stearate, talc, sugar, lactose or a combination thereof.

The choice of the formulation depends on the intended route of administration, such as oral, intravenous, intraperitoneal or subcutaneous administration, or local administration via tumor injection.

Other than oral administration, the pharmaceutical combination of metformin and statin can also be administrated via a route outside the gastrointestinal tract, for example, intravenous or intraarterial injection, or subcutaneous, intramuscular, intrathecal, intraperitoneal, intrarectal, viginal, nasal, intragastric, intratracheal, pulmonary, intratumoral or peritumoral injection or implantation. On the other hand, when the cancer therapy is, for example, a chemical treatment, all the routes of administration stated above may be used as administration routes of the pharmaceutical combination and/or a therapeutically active composition of the cancer therapy.

The pharmaceutical composition of the pharmaceutical combination may be provided as sustained release or timed release formulations. The carrier or diluent may include any sustained release material known in the art, such as glyceryl monostrearate or glyceryl distearate, alone or mixed with a wax. Controlled release preparations can be achieved by the use of polymers to complex or adsorb the cancer therapy sensitizer and/or chemotherapeutic agent. The controlled delivery can be realized by selecting appropriate macromolecules (for example, polyesters, polyamino acids, polyvinyl pyrrolidone, ethylenevinylacetate, methylcellulose, carboxymethylcellulose, and protamine sulfate) and appropriate concentrations of the macromolecules as well as the appropriate methods of incorporation in order to control release. Microencapsulation may also be used. The timed release formulation can provide a combination of immediate and pulsed release throughout the day.

A combination of metformin and statin is preferred, but not limited, to be used with a radiation treatment or a chemical treatment, which are demonstrated individually hereunder as representative examples.

Radiotherapy may be used to treat almost all types of cancer. Radiation may come from a machine outside of the body, known as external radiation, may be placed inside the body, known as internal radiation, or may be unsealed radioactive materials that travel through the body, known as systemic radiation therapy. The type of radiation to be given depends on the type, size, and location of cancer, how close the cancer is to normal tissues that are sensitive to radiation, how far into the body the radiation needs to travel, general health and medical history of patients, whether the patients are receiving other types of cancer treatment, and other factors. In the present disclosure, the pharmaceutical combination may be used as a sensitizer for simultaneously or sequentially administering with a radiotherapy to a subject suffering from cancer.

In certain examples, the radiotherapy herein is an external radiation therapy. More specifically, the radiotherapy is selected from a group consisting of 2DXRT, 3D conformal radiation therapy (3DCRT), intensity-modulated radiation therapy (IMRT), image-guided radiation therapy (IGRT), stereotactic radiosurgery (SRS), stereotactic ablative radiotherapy (SABR), intraoperative radiation therapy (IORT), proton therapy, and other particle therapies.

Radiotherapy according to the embodiments of the present disclosure may comprise providing a cumulative external irradiation to a subject in a dose of 1 to 100 Gray (Gy). A preferred range of the irradiation dose is 1 to 80 Gy. In certain examples, the dose of radiation therapy is less than 90 Gy, such as less than 80 Gy, less than 70 Gy, less than 60 Gy, less than 50 Gy, less than 40 Gy, less than 30 Gy, or less than 20 Gy. In certain examples, the dose of radiation therapy is in a range of about 10 to 100 Gy, such as about 20 to 90 Gy, or about 40 to 80 Gy. In certain examples, the irradiation dose is selected from 5-30 Gy, such as from 8-20 Gy.

An external irradiation dose may be administered in 1 to 100 fractional doses, such as 5 to 80 fractional doses. In certain examples, the fractional doses are administered with about 1.5 Gy to about 3 Gy per fraction, such as about 1.5 Gy, about 1.6 Gy, about 1.7 Gy, about 1.8 Gy, about 1.9 Gy, about 2.0 Gy, about 2.1 Gy, about 2.2 Gy, about 2.3 Gy, about 2.4 Gy, about 2.5 Gy, about 2.6 Gy, about 2.7 Gy, about 2.8 Gy, about 2.9 Gy, or about 3 Gy per fractional dose.

Fractional doses of a radiation therapy may be administered at intervals. In certain examples, the fractional doses are administered over a period of minutes, hours, days, or weeks, such as 1 to 26 weeks, 1 to 15 weeks, or 2 to 12 weeks. In certain examples, the fractional doses are administered over a period of less than about 15 weeks, such as less than 10 weeks, less than 5 weeks, or less than 1 weeks. In certain examples, the cumulative external irradiation is a therapeutically effective amount of radiation for killing cells.

In other examples, the radiation therapy is administered in a single dose rather than in fractional doses. For example, the single dose may be administered with about 1-40 Gy per dose, such as 5-30 Gy or 8-20 Gy. In certain examples, a radiation sensitizer is administered to a subject and the subject is treated by a single dose of radiation therapy within 10 minutes, within 20 minutes, within 30 minutes, within 40 minutes, within 50 minutes or within an hour of the administration of the sensitizer.

In other examples, the radiotherapy is an internal radiation therapy, such as interstitial radiation therapy, intracavitary radiation therapy, or episcleral radiation therapy. In other examples, the radiotherapy is a systemic radiation therapy.

When applying the embodiments of the present disclosure to a radiation treatment, a pharmaceutical combination of metformin and statin can be provided together with one or more effective dose of radiation to an individual in need to improve or enhance the effect of an equal dose of the same radiation upon a cancer or a tumor in an individual in need. In the embodiments of the present disclosure, a cancer therapy sensitizer can be applied before, during or both before and during the application of radiation.

In certain examples, a cancer therapy sensitizer of the pharmaceutical combination is provided before the administration of every dose of radiation, or a cancer therapy sensitizer is re-administrated during the period of every two, three or five times of administration of a dose of radiation. In other examples, only one administration of a cancer therapy sensitizer is required. However, the present disclosure is not limited thereto.

In present disclosure, the pharmaceutical combination may be used as a sensitizer for simultaneously or sequentially administering with a chemotherapeutic agent to a subject suffering from cancer.

The chemotherapeutic agent herein may be a variety of agents, including alkylating agents, platinum compounds, anthracyclines, pyrimidine and purine analogues or antagonists, inhibitors of nucleoside diphosphate reductase, BLM-group antibiotics, inhibitors of topoisomerase, inhibitors of histonedeacetylase, anti-mitotic agents, inhibitors of cell-cycle, inhibitors of proteasome, gestagens, estrogens, anti-androgens, aromatase inhibitors, steroids, gonadotropin-releasing hormone (GnRH) analogues, anti-estrogens, luteinizing hormone-releasing hormone (LHRH) antagonist, and inhibitors of tyrosine kinase receptor; but the present invention is not limited thereto. The choice of chemotherapeutic agent depends on the types of cancer, general health condition and medical history of the patient, whether the patient will receive other types of cancer treatment, and other factors.

More specifically, for example, the therapeutic agent may be selected from the group consisting of melphalan, cyclophosphamide, oxazaphosphorines, cisplatin, carboplatin, oxaliplatin, satraplatin, tetraplatin, iproplatin, mitomycin, streptozocin, carmustine, lomustine, busulfan, ifosfamide, streptozocin, thiotepa, chlorambucil, nitrogen mustards (such as mechlorethamine), ethyleneimine compounds, alkylsulphonates, daunorubicin, doxorubicin (adriamycin), liposomal doxorubicin (doxil), epirubicin, idarubicin, mitoxantrone, amsacrine, dactinomycin, distamycin, netropsin, leucovorin, pibenzimol, duocarmycins, mithramycin, chromomycin, olivomycin, phtalanilides (propamidine, stilbamidine), anthramycins, aziridines, nitrosoureas, cytarabine, 5-fluorouracile (5-FU), uracil mustard, fludarabine, gemcitabine, capecitabine, mercaptopurine, cladribine, azaribine, thioguanine, methotrexate, pentostatin, bryostatin-1, hydroxyurea, folic acid, phleomycins, bleomycins, liblomycin, acridines, rifamycins, actinomycins, adramycin, camptothecin, irinotecan (Camptosar), topotecan, amsacrines, tricyclic carboxamides, suberoylanilide hydroxamic acid (SAHA), trichostatin A, m-carboxycinnamic acid bis-hydroxamide (CBHA), LAQ824, valproic acid, taxanes (such as paclitaxel or docetaxel), vinca alkaloids (such as vinblastine, vincristine, vindesine, vinorelbine (Navelbine), desoxyvincaminol, vincaminol, vincamajine, vineridine, vinburnine), tropolone alkaloids (such as colchicine), macrolides (such as maytansine, ansamitocins, rhizoxin), phomopsin, dolastatin, epipodophyllotoxins, dacarbazine, etoposide, teniposide, steganacins, combretastatin, amphetinile, procarbazine, bortezomib (Velcade™), estramustine, megestrol, flutamide, casodex, anandron, cyproterone acetate, hydroxyprogesterone caproate, medroprogesterone acetate, testosterone propionate, phenyl butyrate, amonogluthetimide, thalidomide, anastrozole, formestan, letrozole, leuprorelin, buserelin, goserelin, triptorelin, dexamethasone, prednisone, fluoxymesterone, prednisolone, methylprednisolone, budenoside, fluocortolone, triamcinolone, diethylstilbestrol, ethinyl estradiol, tamoxifen, droloxifene, trioxifene, raloxifene, zindoxifene, aminoglutethimide, formestane, fadrozole, finasteride, ketoconazole, leuprolide, L-asparaginase, atrasentan, rituximab, cetuximab, bevacizumab (Avastin™), IMC-1C11, Cetuximab (Erbitux), DC-101, matuzumab, vitaxin, imatinib, and derivatives or pharmaceutical acceptable salts thereof.

When applying the embodiments of the present invention to a chemical treatment, the pharmaceutical combination of metformin and statin may be administrated together with one or more effective amount of chemotherapeutic agent to improve or enhance the effectiveness of the chemotherapeutic agent on a cancer or a tumor in an individual in need. According to some embodiments of the present disclosure, when used as a cancer therapy sensitizer, the pharmaceutical combination of metformin and statin may be applied before, during or both before and during a chemotherapy treatment procedure. To be specific, the cancer therapy sensitizer may be administrated with the chemotherapeutic agent at the same time or at a different time of the day, for example, with an interval of 1 or 5 hours.

In certain examples, when the pharmaceutical combination of metformin and statin is used as a cancer therapy sensitizer, the pharmaceutical combination and a chemotherapeutic agent are produced independently and thus can be administrated independently. In other examples, the pharmaceutical combination can also be produced together with the chemotherapeutic agent into a pharmaceutical composition; that is, this pharmaceutical composition contains a single dosage of the cancer therapy sensitizer and an effective amount of the chemotherapeutic agent in the form of a tablet or capsule to facilitate treatment of specific cancers. In the present disclosure, the ratio of the cancer therapy sensitizer over the chemotherapeutic agent may vary according to the subject receiving the treatment, and physiological condition and type of cancer thereof. As for the method of preparing the pharmaceutical composition, all dosage form and other ingredients other than the cancer therapy sensitizer can be prepared by one with ordinary skill in the art according to the present disclosure.

In other examples according to the embodiments of the present disclosure, when applied to sensitize a chemotherapy treatment, the pharmaceutical combination may further be combined with other treatments, such as a radiotherapy.

In the present disclosure, when used as a cancer therapy sensitizer, the pharmaceutical combination of metformin and statin is used before, during or both before and during the application of a cancer therapy. The combination of metformin and statin can cause a synergistic effect to improve or enhance the effect of one or more cancer therapeutic methods on a cancer or a tumor in an individual in need, thus achieving the effects of eliminating, inhibiting, improving, alleviating or preventing a cancer and symptoms thereof; retarding, prohibiting, reversing the rate of tumor proliferation. Alternatively, the combination of metformin and statin can cause medical effects similar to the aforementioned effects, especially to improve, enhance or intensify the cytotoxicity of radiotherapy or chemotherapy to a cancer, a cancer cell, a variant cell, a tumor cell or a combination thereof, to decrease resistance of a cancer cell or various type of the aforementioned cells to cancer therapeutic method, to enhance apoptosis of a cancer cell or various type of the aforementioned cells initiated by the radiotherapy or the chemotherapy, or the combination of the above. The aforementioned cancer therapeutic methods may include, but are not limited to, the step of providing an effective dose of radiation or providing an effective amount of chemotherapeutic agent.

Diseases to be treated by the methods and pharmaceutical combination of the embodiments of the present disclosure may be all kinds of proliferative diseases including cancers and other diseases such as neoplasias and hyperplasias. In certain examples, the disease is selected from solid tumor, such as adrenal cancer, anal cancer, bile duct cancer, bladder cancer, bone cancer, brain or central nervous system (CNS) tumor, breast cancer, Castleman disease, cervical cancer, colorectal cancer, endometrial cancer, esophagus cancer, Ewing family of tumors, eye cancer, gallbladder cancer, gastrointestinal carcinoid tumors, gastrointestinal stromal tumor, gestational trophoblastic disease, Hodgkin disease, Kaposi sarcoma, kidney cancer, laryngeal and hypopharyngeal cancer, liver cancer, lung cancer, lung carcinoid tumor, lymphoma, malignant mesothelioma, nasal cavity and paranasal sinus cancer, nasopharyngeal cancer, neuroblastoma, non-Hodgkin lymphoma, oral cavity and oropharyngeal cancer, osteosarcoma, ovarian cancer, and pancreatic cancer, penile cancer, pituitary tumors, prostate cancer, retinoblastoma, rhabdomyosarcoma, salivary gland cancer, skin cancer, small intestine cancer, stomach cancer, testicular cancer, thymus cancer, thyroid cancer, uterine sarcoma, vaginal cancer, vulvar cancer, Waldenstrom macroglobulinemia and Wilms tumor, and hematologic neoplasias, such as leukemia, multiple myeloma, myelodysplastic syndrome. Preferably, the disease is selected from the group consisting of breast cancer, cervical cancer, colorectal cancer, liver cancer, lung cancer, pancreas cancer and stomach cancer.

### EXPERIMENTAL EXAMPLE

Lewis lung carcinoma (LLC) model was established. In brief, C57BL/6 mice were injected subcutaneously with 1×10⁶ Lewis lung carcinoma cells that constitutively express the firefly luciferase gene in the right thigh.

The aforementioned mice were randomized to eight groups (n=10/group) and treated as follows: Group 1 (sham) did not receive any sensitizer administration or irradiation treatment, and acted as a negative control; Group 2 (sham+M) were administered with 200 mg/kg/day of metformin (Loditon®) without irradiation treatment; Group 3 (sham+S) were administered with 80 mg/kg/day of fluvastatin (Lescol®) without irradiation treatment; Group 4 (sham+M+S) were administered with 200 mg/kg/day of metformin and 80 mg/kg/day of fluvastatin without irradiation treatment; Group 5 (RT) received 40 Gy of irradiation in two 20 Gy fractions; Group 6 (RT+M) were administered with 200 mg/kg/day of metformin combined with 40 Gy of irradiation treatment in two 20 Gy fractions; Group 7 (RT+S) were administered with 80 mg/kg/day of fluvastatin combined with 40 Gy of irradiation treatment in two 20 Gy fractions; and Group 8 (RT+M+S) were administered with 200 mg/kg/day of metformin and 80 mg/kg/day of fluvastatin combined with 40 Gy of irradiation treatment in two 20 Gy fractions.

The day of the LLC injection was defined as day 0. Drug administrations for Group 2 (sham+M), Group 3 (sham+S), Group 4 (sham+M+S), Group 6 (RT+M), Group 7 (RT+S), and Group 8 (RT+M+S) started from day 7 to day 11. The two fractions of radiation treatment was given at day 8 and day 10 respectively to Group 5 (RT), Group 6 (RT+M), Group 7 (RT+S), and Group 8 (RT+M+S). The mice in different Groups were sacrificed at day 21 for further analysis.

### EXAMPLE 1

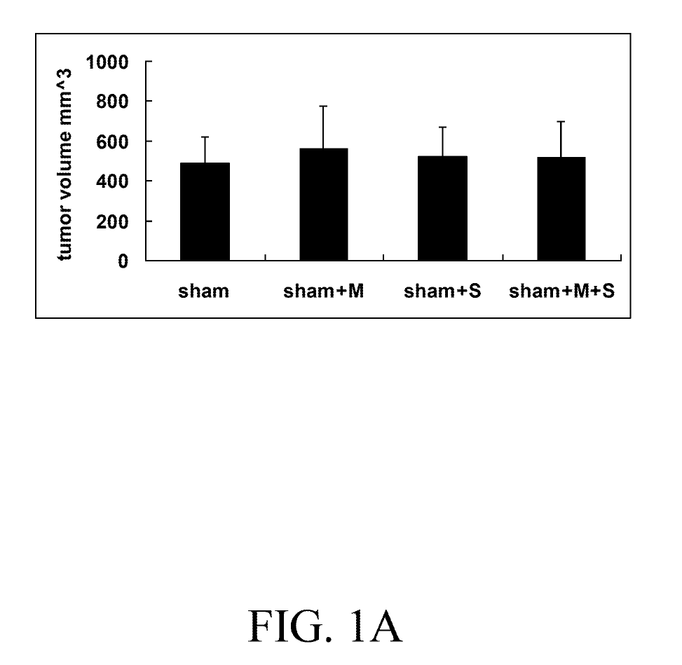

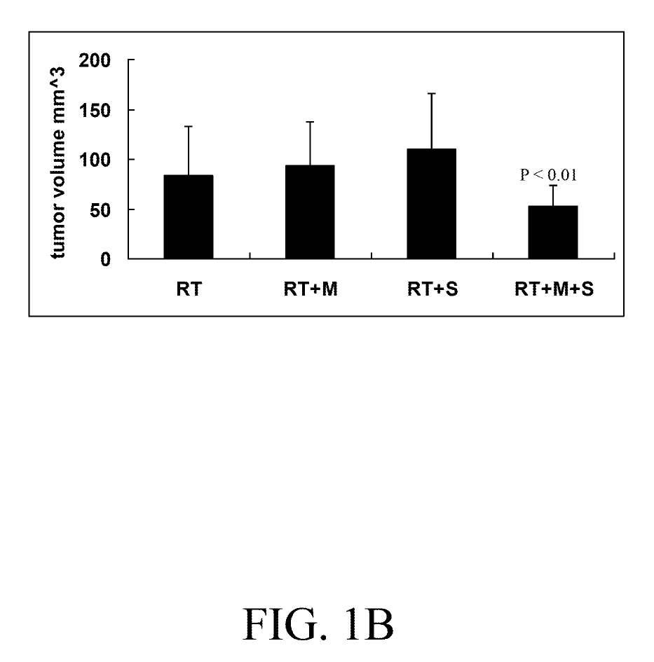

The cancer inhibiting effect of metformin and statin was examined. After the mice were sacrificed, tumor volume on the right thigh of the mice was measured to deteiiiiine local tumor growth. As showed in FIG. 1A, the data revealed that the tumor volumes, as compared with the sham group, revealed that single uses of metformin, statin and metformin+statin did not exhibit any therapeutic effect.

### EXAMPLE 2

As showed in FIG. 1B the tumor volumes were smaller in all of the RT, RT+M, RT+S and RT+M+S groups, as compared with the sham group in FIG. 1A. Moreover, tumor volume of the mice treated with the combination of metformin and statin (RT+M+S) were significantly smaller than tumor volume of RT alone, RT+M and RT+S, showing a P value lower than 0.01. The result shows that the combination of metformin and statin enhanced local tumor-suppressive effect of radiotherapy by 37% in 14 days after completion of the radiation therapy.

### EXAMPLE 3

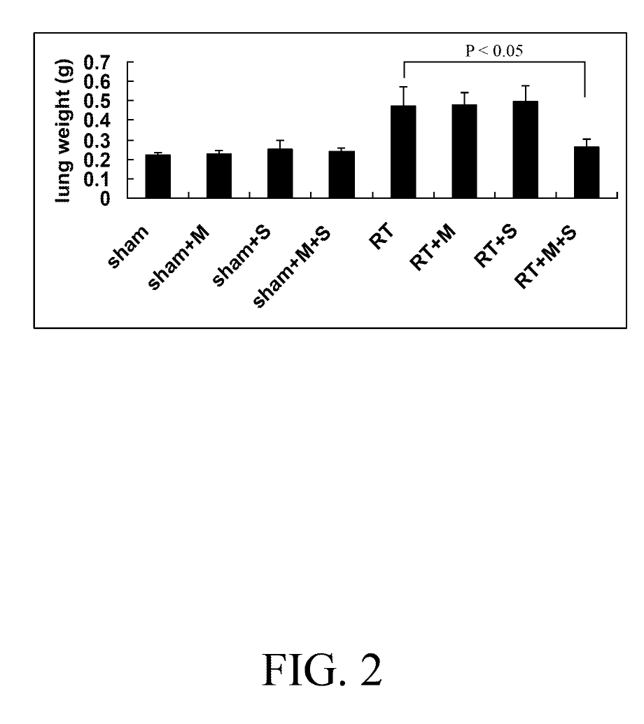

It has been reported that radiotherapy may enhance cancer metastasis (Kevin Camphausen et al. Cancer Research, Vol. 61, No. 5, pp. 2207-11, 2001 and C. H. Chou et al. Oncogene, Vol. 31, No. 4, pp. 458-68, 2012). Therefore, lung metastasis was also examined in our LLC model. Lung weights of the mice were measured after the mice were sacrificed at day 21. The result as showed in FIG. 2 indicated that the lung weights were increased from 0.22 g (ranging 0.21-0.24 g) in the sham to 0.47 g (ranging 0.36-0.67) in the RT alone group. Furthermore, RT with the combination of metformin and statin treatment reduced lung metastasis. The lung weight in the RT+M+S group averaged at 0.26 g and was 18.1% lower as compared with the RT alone group, showing a P value lower than 0.05. The average number of lung surface metastases were increased from 2.4 (ranging 0-5) in the sham group to 18.5 (ranging 12-38) in the RT alone group. The RT+M+S group reduced the radiation induced lung surface metastasis by 22% (with an average number of 10.2) as compared with the RT alone group, showing a P value lower than 0.05.

Although the invention has been described with reference to specific examples, this description is not meant to be construed in a limiting sense. Various modifications of the disclosed examples, as well as alternative examples, will be apparent to persons skilled in the art. It is, therefore, contemplated that the appended claims will cover all modifications that fall within the true scope of the invention.

What is claimed is:

1. A pharmaceutical combination for sensitizing a subject to a cancer treatment, comprising:
an amount of metformin; and
an amount of statin,
wherein the amount of metformin and the amount of statin together constitute an effective amount of the pharmaceutical combination.

2. The pharmaceutical combination according to claim 1, wherein the statin is selected from a group consisting of atorvastatin, fluvastatin, lovastatin, pravastatin, rosuvastatin, and simvastatin.

3. The pharmaceutical combination according to claim 1, wherein the cancer treatment is for lung cancer.

4. The pharmaceutical combination according to claim 1, wherein the cancer treatment is a radiation therapy.

5. The pharmaceutical combination according to claim 4, wherein the radiation therapy is administered in fractional doses.

6. The pharmaceutical combination according to claim 5, wherein a cumulative dose of the fractional doses is within a range of 40-80 Gy.

7. The pharmaceutical combination according to claim 4, wherein the radiation therapy is administered in a single dose.

8. The pharmaceutical combination according to claim 7, wherein the single dose is within a range of 5-30 Gy.

9. The pharmaceutical combination according to claim 1, wherein a weight percentage ratio of the amount of metformin over the amount of statin is within a range of 1:1 to 1000:1.

10. The pharmaceutical combination according to claim 1 being included in a pharmaceutical composition.

11. A method of treating cancer, comprising
administering a pharmaceutical combination comprising an amount of metformin and an amount of statin to a subject, wherein the amount of metformin and the amount of statin together constitute an effective amount of the pharmaceutical combination; and
delivering an effective dose of radiation to the subject.

12. The method according to claim 11, wherein the statin is selected from a group consisting of atorvastatin, fluvastatin, lovastatin, pravastatin, rosuvastatin, and simvastatin.

13. The method according to claim 11, wherein the cancer is lung cancer.

14. The method according to claim 11, wherein the effective dose of radiation is administered in fractional doses.

15. The method according to claim 14, wherein a cumulative dose of the fractional doses is within a range of 40-80 Gy.

16. The method according to claim 11, wherein the effective dose of radiation is administered in a single dose.

17. The method according to claim 16, wherein the single dose is within a range of 5-30 Gy.

18. The method according to claim 11, wherein a weight percentage ratio of the amount of metformin over the amount of statin is within a range of 1:1 to 1000:1.

19. The method according to claim 11, wherein the pharmaceutical combination is included in a pharmaceutical composition.
